(19) United States
(12) Patent Application Publication
(10) Pub. No.: US 2018/0155798 A1
(43) Pub. Date: Jun. 7, 2018

(54) In-Vitro Detection of Human Parvovirus 4

(71) Applicants: Indian Council of Medical Research, New Delhi (IN); King George's Medical University, Lucknow (IN)

(72) Inventors: Amita Jain, Lucknow (IN); Prakash Shantanu, Lucknow (IN)

(21) Appl. No.: 15/524,367

(22) PCT Filed: Nov. 4, 2015
(86) PCT No.: PCT/IN2015/000409

§ 371 Date: May 4, 2017

(30) Foreign Application Priority Data
Nov. 5, 2014 (IN) 3201/DEL/2014

(51) Int. Cl. C12Q 1/70; C12Q 1/6806; C12N 15/09; C12Q 1/686

(52) U.S. Cl. CPC C12Q 1/701; C12Q 1/6806; C12N 2750/14011; C12Q 1/686; C12N 15/09

(57) ABSTRACT

Provided herein are primers and probes for in-vitro detection of human parvovirus 4, including primers and probes having the sequence of SEQ ID NO: 1 or a part of it or a nucleotide having minimum 90% sequence identity with SEQ ID NO: 1; SEQ ID NO: 2 or a part of it or a nucleotide having minimum 90% sequence identity with SEQ ID NO: 2; and/or SEQ ID NO: 3 or a part of it or a nucleotide having minimum 90% sequence identity with SEQ ID NO: 3. The nucleotide sequences enable high detection of all genotypes of human parvovirus 4.

### FIELD OF INVENTION

The present invention relates to the field of in vitro detection of Human Parvovirus 4 in human plasma, serum, CSF, nasal and throat swab samples. More particularly, the invention relates to highly sensitive and specific probes and primers for Real Time PCR which enables the detection of Human Parvovirus 4. The present invention also provides probes and primers that advantageously helps in detecting all the genotypes & subtypes of Human parvovirus 4 and also provided probes and primers that are efficient enough to detect sample with low copy numbers of virus. Further, the invention extends to provide a reaction mixture for the realtime PCR which enables the detection of Human parvovirus 4.

### BACKGROUND AND PRIOR ART OF THE INVENTION

Human Parvovirus 4 (Parv4) is a new virus in the Parvoviridae family, discovered in 2005 in blood from an intravenous drug user (IVDU). Human Parvovirus 4 is a single-stranded DNA virus, 5.3 kb in length, nonenveloped, spherical particles with diameter of 20-25 nm. The genome comprises two main open reading frames, but unlike Human parvovirus B19, the two ORFs do not overlap. It is expected that due to similarity to Human Parvovirus B19 and other parvoviruses it might have similar properties.

Parvovirus 4 and Parvovirus 4-like viruses from other primates and swine have been categorized together in a new genus, Partetravirus. Parv4 genotypes 1 and 2 have been detected in the northern hemisphere while genotype 3 was discovered in Africa. In the northern hemisphere Parv4 DNA and Parv4 antibodies are found mostly in IVDUs and others who are parenterally exposed.

In Africa Parv4 infection seems to be more widespread. The pathogenicity of Parv4 is unknown, but Parv4 has been linked to meningitis in children, hydrops fetalis and has also been found in coinfection with Human immunodeficiency virus, Hepatitis C virus and Hepatitis B virus. Exact route of transmission is yet to be established but respiratory, transplacental & parenteral routes are implicated in causing the spread.

The increasing incidence and possible association of Human parvovirus 4 with many of the clinical disease raises the demand for a highly sensitive, specific kit for detection of human parvovirus 4. There is no FDA approved currently used methods for the diagnosis of human parvovirus 4 however few PCR assays and IgG and IgM antibody tests have been developed by academic laboratories. Moreover due to genotypic diversity many of the assays have limitations in picking up all the existing genotypes. The Real Time PCR based assays for the detection of human parvovirus 4 nucleic acids in the human plasma, serum, CSF, nasal and throat swab samples of an infected subject may provide an advantage.

EP 2251441 A1 (Stefan Schorling, 2010) discloses a detection method and kit for detecting Human parvovirus B19 in a test sample. However, the test does not detect the presence of human parvovirus 4.

WO 2003002753 a2 (Sergio Pichuantes, Venkatakrishna Shyamala, 2003) describes a method for detection of detecting human parvovirus b19 in a test sample. However, this test also does not detect the presence of human parvovirus 4.

The patents US 2014106337(A1) & US 2003170612(A1) provides a detection system for human parvovirus B 19 detection which has nothing to do with human parvovirus 4. Human parvovirus 4 has no genome similarity with human parvovirus B 19 and both are different viruses. However, the paper entitled “Analysis of two human parvovirus PARV4 genotypes identified in human plasma for fractionation” is based on conventional PCR which has already known many limitations over Real Time PCR. Another paper “A two-step real-time PCR assay for quantitation and genotyping of human parvovirus 4” (Vaisanen et al; 2014) has similar technique as used by the present invention but the primer and probes used in this paper has many limitation in context to sensitivity in the detection of human parvovirus 4.

Hence, there is no appropriate method for detection of human parvovirus 4. It is therefore desirable to provide a method of detection of the target nucleic acid in a sample using real time polymerase chain reaction that overcomes the above-mentioned disadvantages. Further there is a need to provide a highly sensitive and specific testing kit which enables the detection of human parvovirus 4 virus overcoming the problem of missing any case i.e., which can help in detecting all the genotypes of Human Parvovirus 4. There is also a need of a test kit which helps in detecting the samples infected with low copy number of Human Parvovirus 4.

### OBJECT OF THE INVENTION

It is therefore, an object of the present invention is to provide probes and primers for the detection of Human parvovirus 4.

Another object of the present invention is to provide a highly sensitive and specific testing kit using the aforesaid probes and primers which enables the detection of human parvovirus 4 overcoming the problem of missing any case.

Another object of the present invention is to provide a test kit which can help in detecting all the genotypes of Human Parvovirus 4.

Another object of the present invention is to provide a test kit which helps in detecting the sample with low copy number virus.

Yet another object of the present invention is to provide a reaction mixture for the amplification of human parvovirus 4 which enables the specific and sensitive of detection of human parvovirus 4.

### SUMMARY OF THE INVENTION

According to this invention, there is provided primers and probes for in-vitro detection of human parvovirus 4, the probes and primers comprises of nucleotide sequences selected from the group comprising of or any combinations thereof:
a) a nucleic acid molecule that encodes a nucleotide sequence of SEQ ID NO:1 or a part of it or a nucleotide having minimum 90% sequence identity with SEQ ID NO:1;
b) a nucleic acid molecule that encodes a nucleotide sequence of SEQ ID NO:2 or a part of it or a nucleotide having minimum 90% sequence identity with said SEQ ID NO:2;
c) a nucleic acid molecule that encodes a nucleotide sequence of SEQ ID NO:3 or a part of it or a nucleotide having minimum 90% sequence identity with said SEQ ID NO:3;
   wherein the nucleotide sequences enable high detection of all genotypes of human parvovirus 4.

The present invention further provides a reaction mixture for real time PCR comprising:
nuclease free water present in an amount of 5.625 μl;
buffer present in an amount of 12.5 μl;
human parvovirus 4 Primer Forward (10 pm) present in an amount of 0.35 μl;
human parvovirus 4 Primer Reverse (10 pm) present in an amount of 0.35 μl;
human parvovirus 4 Probe (10 pm) present in an amount of 0.175 μl;
enzyme present in an amount of 1.0 μl; and
template present in an amount of 5 μl;
   wherein the reaction mixture enables equal intensity detection of all genotypes of Human Parvovirus 4.

### BRIEF DESCRIPTION OF THE ACCOMPANYING DRAWINGS

FIG. 1 shows the alignment of Human parvovirus 4 primers and probe with VP1 region of all three genotype of Human parvovirus 4.

FIG. 2 shows a Real time plot of Human parvovirus 4 positive samples using SEQ ID No. 1, 2 &3 A showing Human parvovirus 4 amplification and B line showing No Template Control.

FIG. 3 shows real time plot of HUMAN PARVOVIRUS 4 tested in clinical samples using SEQ ID No. 1, 2&3 A showing HUMAN PARVOVIRUS 4 DNA positive samples and B line showing negative samples & NTC.

### DETAILED DESCRIPTION OF THE INVENTION

The present invention provides a real-time polymerase chain reaction for detection of Human parvovirus 4 together with a new set of primers and probes with high sensitivity and specificity.

The invention further provides a test kit based on real time PCR for the detection of Human Parvovirus 4, said kit comprises nucleotide sequences selected from the group comprising of or any combinations thereof:
a) a nucleic acid molecule that encodes a nucleotide sequence of SEQ ID NO:1 or a part of it or a nucleotide having at least 90% sequence identity with said SEQ ID NO:1; (Please check if 90% sequence identity is appropriate)
b) a nucleic acid molecule that encodes a nucleotide sequence of SEQ ID NO:2 or a part of it or a nucleotide having at least 90% sequence identity with said SEQ ID NO:2; (Please check if 90% sequence identity is appropriate)
c) a nucleic acid molecule that encodes a nucleotide sequence of SEQ ID NO:3 or a part of it or a nucleotide having at least 90% sequence identity with said SEQ ID NO:3; (Please check if 90% sequence identity is appropriate)
   wherein said nucleotide sequences enable high detection of all genotypes of human parvovirus 4.
   The nucleotide sequences enable detection of Human Parvovirus 4 present in low copy (number up to ≥100 copies/ml.)

The invention also provides primers and probes for the detection of Human Parvovirus 4 virus in a sample comprising:
a) a nucleic acid molecule that encodes a nucleotide sequence of SEQ ID NO:1 or a part of it or a nucleotide having at least 90% sequence identity with said SEQ ID NO:1, wherein said nucleotide sequence of SEQ ID NO: 1 represents forward primer to amplify Human Parvovirus 4;
b) a nucleic acid molecule that encodes a nucleotide sequence of SEQ ID NO:2 or a part of it or a nucleotide having at least 90% sequence identity with said SEQ ID NO:2, wherein said nucleotide sequence of SEQ ID NO: 1 represents reverse primer to amplify Human Parvovirus 4;
c) a nucleic acid molecule that encodes a nucleotide sequence of SEQ ID NO:3 or a part of it or a nucleotide having at least 90% sequence identity with said SEQ ID NO:3, wherein said nucleotide sequence of SEQ ID NO:3 represents probes to detect Human Parvovirus 4;
   wherein said nucleotide sequences enable high detection of all genotypes of human parvovirus 4.

The nucleotide sequences enable detection of Human Parvovirus 4 present in low copy (number up to ≥100 copies/ml.)

The invention further provides a reaction mixture for real time PCR for the detection of Human Parvovirus 4 virus comprising:
a) Nuclease free water present in an amount of 5.625 μl.
b) Buffer present in an amount of 12.5 μl.
c) Human parvovirus 4 Primer Forward (10 pm) present in an amount of 0.35 μl.
d) Human parvovirus 4 Primer Reverse (10 pm) present in an amount of 0.35 μl.
e) Human parvovirus 4 Probe (10 pm) present in an amount of 0.175 μl.
f) Enzyme present in an amount of 1.0 μl.
g) Template present in an amount of 5 μl.

The reaction mixture advantageously enables equal intensity detection of all genotypes of Human Parvovirus 4 virus.

The reaction mixture further advantageously enables detection of human parvovirus 4 with high sensitivity and also of all the genotypes of Human Parvovirus 4.

Designing the Primers and Probes for Human Parvovirus 4

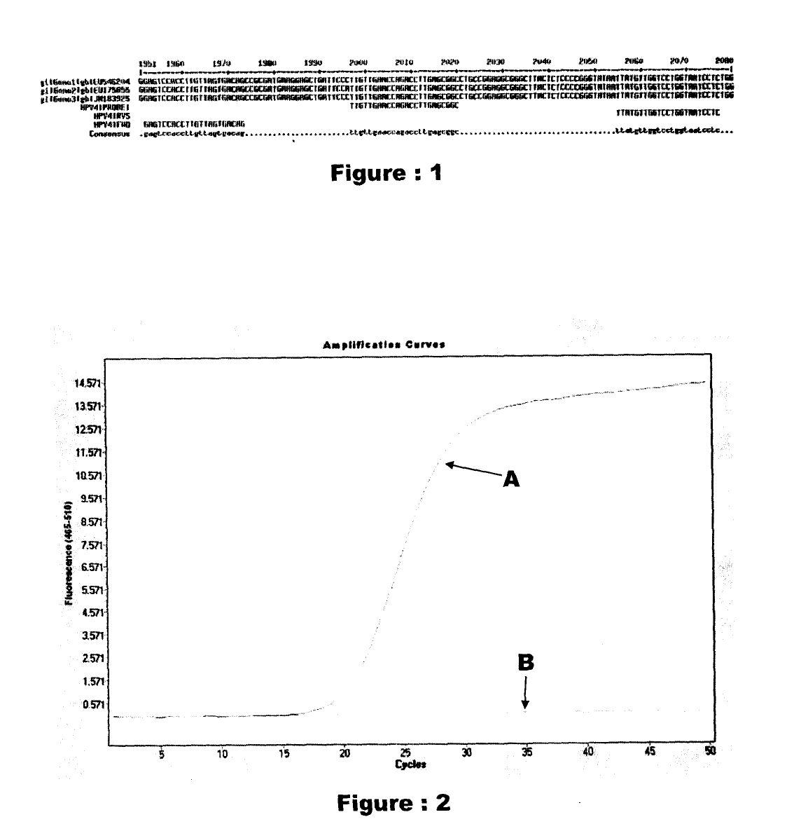

Sequences representing all three Human parvovirus 4 genotypes (1-3) accession no. (genotype 1: EU546204.1, genotype 2: EU175855.1, genotype 3: JN183925.1) and many other sequences representing Human parvovirus 4 were downloaded from the GenBank nucleotide database and aligned using the program Mega5.1. A highly conserved region of the VP1 gene is selected for the design of real-time PCR primers and probe. All three Human parvovirus 4 genotypes which we studied are presented in FIG. 1. Primer and probe were designed in such a way that there should not be any degeneracy among the primers or probes making it more stable and also the primers and probe suitable for all the genotypes. The probe is tagged with FAM as a reporter and IABkFQ as a quencher at 3′ end. The primers and probes sequences for Human parvovirus 4 are mentioned in table 1:

TABLE 1

| Sequence | | Oligo- | | Product |
|---|---|---|---|---|
| ID | Polarity | nucleotide sequence 5′-3′ | Length | size |
| 1. HPV4 | Forward | GAGTCCACCTTGTTAGTGACAG | 22 | 126 bp |
| Fwd | | | | |
| 2. HPV4 | Reverse | GAGGATTACCAGGACCAACATAA | 23 | |
| Rvs | | | | |
| 3. HPV4 | | /5FAM/TTGTTGAACCAGACCTTG | 24 | |
| Probe | | AGCGGC/3 IABkFQ/ | | |

Although the inventions herein are described with various specific embodiments, it will be obvious for a person skilled in the art to practice the embodiments herein with modifications. However, all such modifications are deemed to be within the scope of the claims. It is also to be understood that the following claims are intended to cover all of the generic and specific features of the embodiments described herein and all the statements of the scope of the embodiments which as a matter of language might be said to fall there between.

Testing the Sensitivity & Specificity of Probes and Primers

A known Human parvovirus 4 sample was amplified with the aforesaid primers and cloned in pTZ57R/T cloning vector using commercial kit then the cloned plasmid was amplified using M13/pUC sequencing primers (Fwd & Rvs), then the amplified product was sequenced using capillary sequencer to get exact cloned sequence. Further the sequenced segment was checked for the sequence similarity using NCBI Blast program and it was confirmed that the cloned sequence belonged to Human parvovirus 4.

A batch of 80 samples was prepared by mixing different viruses in different combinations including Human parvovirus 4 as well as HBV, HCV, Human Parvovirus B19, Adenovirus, Dengue, Japanese encephalitis, HSV-1, HSV-2, Varicella zoster virus and was tested for Human parvovirus 4 by currently designed primer and probes. Results showed that there is no cross reactivity of the designed primers and probes with the above mentioned viruses.

The sensitivity of this Real Time based assay was estimated based on a cloned and standardized parvovirus 4 DNA with known copy number. Detection of positive samples containing 500 copies/ml, 200 copies/ml & 100 copies/ml of the parvovirus 4 standard in the assay was 100% (10 of 10 tests) respectively, while samples with ≤10 copies/ml were tested positive 9 out of 10 times. These results show that the assay has a very high sensitivity and specificity required for detecting human parvovirus 4.

Clinical Specimen Testing

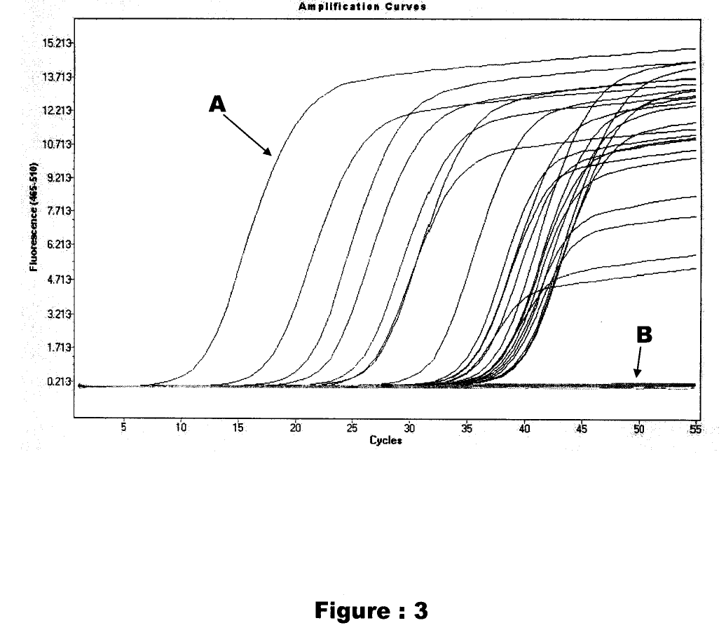

To determine the assay specificity of the primers and probe for detecting Human parvovirus 4 DNA, Real Time-based assay was performed (using amplification oligomers of SEQ ID Nos. 1, 2 and 3) on clinical available serum/plasma samples. The assay was performed on 95 samples from five different subgroups of patients samples stored at virology section, dept. of Microbiology, King George's Medical University, Lucknow, U.P., India. Of total clinical samples tested 33 were positive for the parvovirus 4. All positive samples provided positive results when they were retested three times using the same assay (FIG. 3 showing clinical samples tested for parvovirus 4).

Validation of the Sequence Amplified Using Newly Designed Primers

Few positive samples for human parvovirus 4 were cloned sequenced using ABI 3130 sequencer using M13/pUC sequencing primers. Sequencing results showed all the amplified sequences belonged to human parvovirus 4 (FIG. 3).

In the present embodiment, the invention has been described with reference to detection of Human parvovirus 4 from human plasma, serum, CSF, nasal and throat swab samples, however such description should not be considered as restricting the scope of the present invention. Further it would be possible for a person skilled in the art to practice the present invention considering other sample for the detection of Human parvovirus 4 without departing from the scope of the present invention.

1. A primer or probe for in-vitro detection of human parvovirus 4, wherein the primer or probe comprises the nucleotide sequences selected from the group consisting of:
a) SEQ ID NO: 1 or a sequence having 90% sequence identity with SEQ ID NO: 1;
b) SEQ ID NO: 2 or a sequence having 90% sequence identity with SEQ ID NO:2; and
c) SEQ ID NO: 3 or a sequence having 90% sequence identity with SEQ ID NO: 3;
wherein the nucleotide sequences enable high detection of all genotypes of human parvovirus 4.

2. The primer or probe as claimed in claim 1, wherein the nucleotide sequences enable detection of human parvovirus 4 virus present in low copy number up to ≥100 copies/ml.

3. A reaction mixture for real time PCR comprising:
nuclease free water present in an amount of 5.625 μl;
buffer present in an amount of 12.5 μl;
SEQ ID NO: 1 present in an amount of 0.35 μl;
SEQ ID NO: 2 present in an amount of 0.35 μl;
SEQ ID NO: 3 present in an amount of 0.175 μl;
enzyme present in an amount of 1.0 μl; and
template present in an amount of 5 μl;
wherein the reaction mixture enables equal intensity detection of all genotypes of human parvovirus 4.

4. The reaction mixture as claimed in claim 4, wherein SEQ ID NO: 3 further comprises FAM as a reporter and IABkFQ as a quencher at a 3′end.

5. The primer or probe as claimed in claim 1, wherein SEQ ID NO: 1 and SEQ ID NO: 2 respectively represent forward and reverse primers to amplify human parvovirus 4.

6. The primer or probe as claimed in claim 1, wherein SEQ ID NO: 3 represents a probe for the detection of human parvovirus 4.

7. The primer or probe as claimed in claim 1, wherein the probe or primer for human parvovirus 4 virus is a primer or probe for the VP1 gene of human parvovirus 4.

8. The primer or probe as claimed in claim 1, wherein the primer or probe is sensitive to detect the sample with the viral load of ≥100 copies/ml.

9. A test kit for in-vitro detection of human parvovirus 4, the kit comprises:
a) a nucleic acid molecule having the sequence of SEQ ID NO: 1 or a sequence having 90% sequence identity with said SEQ ID NO: 1;
b) a nucleic acid molecule having the sequence of SEQ ID NO: 2 or a sequence having 90% sequence identity with said SEQ ID NO:2; and/or
c) a nucleic acid molecule having the sequence of SEQ ID NO: 3 or a sequence having 90% sequence identity with said SEQ ID NO:3;
wherein the nucleotide sequence enables high detection of all genotypes of human parvovirus 4.

10. (canceled)


SEQUENCE LISTING (3 sequences)

SEQ ID NO: 1 - DNA, 22 bp, Artificial Sequence
  HPV4 Forward Primer
gagtccaccttgttagtgacag

SEQ ID NO: 2 - DNA, 23 bp, Artificial Sequence
  HPV4 Reverse Primer
gaggattaccaggaccaacataa

SEQ ID NO: 3 - DNA, 24 bp, Artificial Sequence
  HPV4 Probe
ttgttgaaccagaccttgagcggc